(19) United States
(12) Patent Application Publication
(10) Pub. No.: US 2025/0134784 A1
(43) Pub. Date: May 1, 2025

(54) TWO-PHASE MAKEUP REMOVER

(71) Applicant: BEIERSDORF AG, Hamburg (DE)

(72) Inventors: Preksha LAAD, Madhya Pradesh (IN); Saurabh GUPTA, Rajasthan (IN)

(21) Appl. No.: 18/719,360

(22) PCT Filed: Nov. 8, 2022
(86) PCT No.: PCT/EP2022/081130

§ 371 Date: Jun. 13, 2024

(30) Foreign Application Priority Data
Dec. 17, 2021 (IN) 202111058972

(51) Int. Cl. A61K 8/03; A61K 8/33; A61K 8/92; A61Q 1/14

(52) U.S. Cl. CPC A61K 8/03; A61K 8/33; A61K 8/922; A61Q 1/14

(57) ABSTRACT

The present invention belongs to the cosmetic field.

A beautiful and attractive appearance is a desire for many people. One typical sign of such an appearance is a healthy and smooth looking skin. In order to take care on the skin, it is for many people a daily routine to apply decorative cosmetic products such as mascara or foundation. However, before going to bed, most people remove the decorative cosmetic products again. Conventionally the products are removed using a two-phase makeup remover, which has an aqueous phase and an oil phase, which are clearly separated.

The two-phase makeup remover is applied by shaking the product to mix both phases and then transferring some of the mixture onto a suitable tissue. The tissue is then used to cleanse and absorb the makeup from the skin.

The state of the art knows various two-phase makeup remover. Typical examples are described in U.S. Pat. No. 5,165,917A.

A typical ingredient in those products are silicone oils or silicone compounds as well as lower alkanes. Those compounds allow for an effective cleansing as well as a fast and effective phase separation after mixing the ingredients. That means no “sweating” is observed and the phases do not become hazy. The separation time usually takes up to 60 minutes.

In terms of the present disclosure the term “sweating” is understood as oil droplets clinging to the surface of bottle in water phase portion.

In terms of the present disclosure the term “hazy” is understood as not clear and transparent.

A hazy formulation is not clear and transparent.

In terms of the present disclosure the term “lower alkanes” covers all alkanes, which do not have
a higher molecular weight than 480 Da,
a kinematic viscosity at 100° C. of >8.5 cSt, and
a carbon number at 5% boiling point of >25.

This definition is in line with the definition provided in COSMETICS EUROPE RECOMMENDATION N14 17-09-2018 “MINERAL HYDROCARBONS IN COSMETIC LIP CARE PRODUCTS”, available under the link https://www.cosmeticseurope.eu/files/3715/3907/8160/Recommendation_14_Mineral_Hydro_Carbons.pdf.

In terms of the present disclosure the term “silicone oils and silicone compounds” are understood as ingredients comprising a dimethylsiloxane unit.

However, the use of silicone oils and silicone compounds has been controversially discussed in recent years such that many consumers prefer to use products not containing those compounds.

Further the use of lower alkanes is limited by the cited COSMETICS EUROPE RECOMMENDATION N14 17-09-2018 in lip applications. In principle a conventional makeup remover is not used on the lips such that lower alkanes can be included. However, makeup remover may be used closed to sensitive areas such as the eyes and the lips such that it is generally desirable in the interest of the consumer to avoid using these compounds as well.

The absence of the silicone oils, silicone compounds and lower alkanes adversely affects the properties of two-phase makeup remover. Regarding the separation properties the separation time after mixing is often increased. Further sweating effects are often observed. Further the separated phases become more translucent not allowing a clean and pretty appearance to be maintained over the use cycle of the product. Furthermore, the ability to remove waterproof makeup is generally found to be reduced. In particular it is desirable to avoid sweating and haziness of the formulations 24 hours after shaking/mixing the phases.

Accordingly, it was the objective of the present invention to address, solve and/or reduce the shortfalls of the makeup removers containing no silicone oils and lower alkanes.

It was surprisingly found by the application that the objectives can be met by the present invention.

A first object of the present invention is a two-phase cosmetic cleansing composition, which is not an emulsion, comprising
a) An aqueous phase
b) An oil phase
whereby the oil phase is characterized in that
it has an interfacial tension towards water in the range from 22 to 35 mN/m
a density in the range from 0.82 to 0.90 g/cm³,
the oil phase is free from silicone oils and lower alkanes.

Another object of the invention is the use of the composition according to the invention to remove makeup from the human skin.

It was surprisingly noticed that applying the subject matter of the invention sweating and. Accordingly, the objectives of the present invention are solved.

All the weight percentages (% by weight) given below relate, unless otherwise stated, to the total weight of the cosmetic composition. If ratios of certain components are disclosed in the following description, these ratios refer, unless otherwise stated, to weight ratios of the components.

Unless otherwise stated, all tests and measurements were performed under “normal conditions”. The term “normal conditions” refers to 20° C., 1013 hPa and a relative humidity of 60.

To define if an oil is a lower alkane, the following standardized methods are applied:
Kinematic viscosity at 100° C.:
ASTM D-445-21, published 15 May 2021
Carbon number at 5% boiling point:

The specification “carbon number ≥25 at the 5% boiling point” means there is not more than 5% of hydrocarbons with a chain length less than 25. It can be determined by Gas Chromatography. A standard method is ASTM D 6352-19e1 published 1 Dec. 2019

### Molecular Weight:

The mean molecular weight can be determined from the kinematic viscosity of the oil. A standard protocol can be found in ASTM D2502-14(2019)e1 published 1 May 2019.

To define if a wax is a lower alkane, the following standardized methods are applied:

### Viscosity at 100° C.:

The same method as for oils (ASTM D-445) can also be used for waxes. If the material is not totally soluble in the normalized solvent of the method, the viscosity at 100° C. may be obtained by extrapolation from measurements performed at 120° C., 130° C. and 150° C.

### Carbon Number at 5% Boiling Point:

The specification “carbon number ≥25 at the 5% boiling point” means there is not more than 5% of hydrocarbons with a chain length less than 25, which is consistent with food regulations. It can be determined by Gas Chromatography. Standard methods are ASTM D-2887 D2887-19ae2, published 1 Dec. 2019.

### Molecular Weight

The molecular weight is determined by osmometry in four different concentrations in toluene at 65° C.

In the following description the terms “according to the invention”, “preferred according to the invention” and so on are always directed to the cosmetic composition according to the invention, the use according to the invention and to the method according to the invention.

For the purposes of the present disclosure, the term “free from” means that the proportion of the respective substance is less than 0.05% by weight, preferably less than 0.01% by weight and most preferably 0% by weight, based on the total weight of the respective phase of the composition. Accordingly, the % by weight values for silicone oil and lower alkanes corresponds to the total weight of the oil phase. This definition ensures that entrainments or impurities with these substances are not included as “free from” according to the invention.

The term “skin” refers solely to the human skin.

Emulsifiers are understood to be all substances which are listed in the International Cosmetic Ingredient Dictionary and Handbook, Thirteenth Edition 2010, (ISBN 1-882621-47-6) under the name “emulsifying agent”. Surfactants are understood to be all substances which are listed in the International Cosmetic Ingredient Dictionary and Handbook, Thirteenth Edition 2010, (ISBN 1-882621-47-6) under the name “surfactant”.

It is preferred according to the invention if the ratio by volume between the aqueous phase and the oil phase is in the range from 20:1 to 1:20, preferably from 10:1 to 1:2, preferably 8:1 to 2:1, more preferably 7:1 to 3:1 and most preferably 6:1 to 4.5:1.

Further it is preferred if the aqueous phase comprises water in a total quantity of at least 60% by weight, more preferably at least 65% by weight, more preferably at least 70% by weight, more preferably at least 75% by weight, more preferably at least 80% by weight, more preferably at least 85% by weight and most preferably at least 90% by weight, calculated to the total weight of the aqueous phase.

Further it is preferred if the aqueous phase comprises water in a total quantity of less than 99% by weight, more preferably less than 98% by weight, more preferably less than 97% by weight, more preferably less than 96% by weight and most preferably less than 95.5% by weight, calculated to the total weight of the aqueous phase.

The aqueous phase preferably comprises at least one surfactant. The surfactant may be chosen from anionic, nonionic, amphoteric and cationic surfactants, whereby nonionic and amphoteric surfactants are preferred.

Thereby, it is preferred if the total quantity of the surfactants is in the range from 0.01 to 2% by weight, more preferably from 0.02 to 1% by weight, more preferably from 0.025 to 0.5% by weight and most preferably from 0.03 to 0.1% by weight, calculated to the total weight of the aqueous phase.

Preferred anionic surfactants according to the invention include:
linear and branched fatty acids having 8 to 30 C atoms (soaps),
ether carboxylic acids of the formula R—O—(CH₂—CH₂O)ₓ—CH₂—COOH, in which R is a linear or branched, saturated or unsaturated alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and/or dialkyl esters having 8 to 24 C atoms in the alkyl group and sulfosuccinic acid monoalkylpolyoxyethyl esters having 8 to 24 C atoms in the alkyl group and 1 to 6 oxyethyl groups,
alpha-olefinsulfonates having 8 to 24 C atoms,
alkyl sulfate and/or alkyl polyglycol ether sulfate salts of the formula R—(OCH₂—CH₂)ₓ—OSO₃⁻M⁺, in which R is a preferably linear or branched, saturated or unsaturated alkyl group having 8 to 30 C atoms, x=0 or 1 to 12, and M is an alkali or ammonium ion,
sulfonates of unsaturated fatty acids having 8 to 24 C atoms and 1 to 6 double bonds,
esters of tartaric acid and citric acid with alcohols, which are adducts of about 2-15 molecules of ethylene oxide and/or propylene oxide to fatty alcohols having 8 to 22 C atoms,
alkyl and/or alkenyl ether phosphates of the formula,

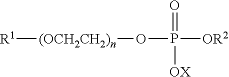

in which R¹ preferably stands for an aliphatic hydrocarbon group having 8 to 30 carbon atoms, R² for hydrogen, a group (CH₂CH₂O)ₙR¹ or X, n for numbers from 0 to 10, and X for hydrogen, an alkali metal or alkaline earth metal, or NR³R⁴R⁵R⁶, where R³ to R⁶ independently of one another stand for a C₁ to C₄ hydrocarbon group.

Preferred anionic surfactants are ether carboxylic acids of the aforementioned formula, acyl sarcosides having 8 to 24 C atoms in the acyl group, sulfosuccinic acid mono and/or -dialkyl esters having 8 to 24 C atoms in the alkyl group and sulfosuccinic acid monoalkylpolyoxyethyl esters having 8 to 24 C atoms in the alkyl group and 1 to 6 oxyethyl groups, alpha-olefinsulfonates having 8 to 24 C atoms, and/or alkylsulfate salts and/or alkyl polyglycol ether sulfate salts of the aforementioned formula.

Especially preferred anionic surfactants are straight-chain or branched alkyl ether sulfates, which include an alkyl group having 8 to 18 and especially having 10 to 16 C atoms, and 1 to 6 and particularly 2 to 4 ethylene oxide units. Furthermore, especially preferred anionic surfactants are straight-chain or branched alkyl sulfonates, which include an alkyl group having 8 to 18 and especially having 10 to 16 C atoms. Preferred in particular are the sodium, magnesium, and/or triethanolamine salts of linear or branched lauryl, tridecyl, and/or myristyl sulfates, which have a degree of ethoxylation of 2 to 4.

Very especially preferred emulsions according to the invention are characterized in that they include at least one anionic surfactant from the group of alkyl sulfates and alkyl polyglycol ether sulfates of the formula R—(OCH₂—CH₂)ₓ—OSO₃M, in which R preferably stands for a straight-chain or branched, saturated or mono- or polyunsaturated alkyl or alkenyl group having 8 to 24 carbon atoms, x for 0 or 1 to 12, and M for an alkali metal or alkaline earth metal or for triethanolamine, whereby anionic surfactants with the INCI name Sodium Lauryl Sulfate are especially preferred.

Preferred amphoteric surfactants can be selected from compounds of the following formulas (i) to (v), in which the group R in each case stands for a straight-chain or branched, saturated or mono- or polyunsaturated alkyl or alkenyl group having 8 to 24 carbon atoms,

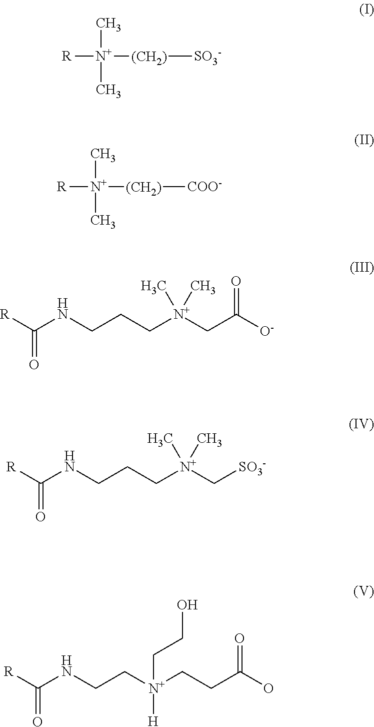

Especially suitable amphoteric surfactants are alkyl amidoalkyl betaines and/or alkyl ampho(di)acetates of the aforementioned formulas (i) to (v). Especially suitable amphoteric surfactants include the surfactants known under the INCI names Sodium Cocoamphoacetate, Cocamidopropyl Betaine and Disodium Cocoamphodiacetate. Most preferred is Sodium Cocoamphoacetate.

Further it is preferred if at least one nonionic surfactant is contained in the aqueous phase. Preferred nonionic surfactants are selected from alkyl glucosides, whereby coco glucoside, caprylyl/capryl glucoside, decyl glucoside and lauryl glucoside are preferred. Most preferred are coco glucoside and/or caprylyl/capryl glucoside.

According to the invention it is further preferred if the aqueous phase comprises at least on complex former, which is preferably EDTA.

Further it is preferred if the aqueous phase of the composition comprises at least one preservative. Preferred preservatives are selected from the group consisting of phenoxyethanol, ethylhexylglycerin and/or benzethonium chloride. Particular preferred are phenoxyethanol and/or benzethonium chloride.

For the case that phenoxyethanol is contained, it is preferred if the total quantity of phenoxyethanol is in the range from 0.1 to 1.5% by weight, more preferably 0.2 to 1.2% by weight and most preferably 0.4 to 0.8% by weight, calculated to the total weight of the aqueous phase.

For the case that benzethonium chloride is contained, it is preferred if the total quantity of benzethonium chloride is in the range from 0.01 to 0.5% by weight, more preferably 0.02 to 0.3% by weight and most preferably 0.04 to 0.2% by weight, calculated to the total weight of the aqueous phase.

A further typical ingredient preferably contained in the aqueous phase is glycerol. For the case that glycerol is contained, it is preferred if the total quantity of glycerol is in the range from 0.1 to 8.0% by weight, more preferably 0.5 to 6.0% by weight and most preferably 1.0 to 5.5% by weight, calculated to the total weight of the aqueous phase.

Further it is preferred if the aqueous phase comprises sodium chloride. Sodium chloride is preferably contained in quantities ranging from 0.01 to 0.6% by weight, more preferably 0.1 to 0.5% by weight and most preferably 0.15 to 0.35% by weight, calculated to the total weight of the aqueous phase.

The oil phase of the composition is characterized in that
it has an interfacial tension towards water in the range from 22 to 35 mN/m
a density in the range from 0.82 to 0.90 g/cm³, and
the oil phase is free from silicone oils and lower alkanes.

According to the invention the interfacial tension of the oil phase is determined using a Tensiometer K100 obtained from the Fa.Krüss.

The parameter for the measurement are described as follows:
Device: Tensiometer K100 (KROSS)
Correction: yes: Density
Temperature: 20° C.

### Setting:

Measurement body: Kruss Standard Ring
Radius: 9,545 mm
Diameter of wire: 0.37 mm

### Parameters:

Velocity Surface Detection: 4 mm/min
Sensitivity Surface-Detection: 0,004 g
Velocity Searching: 4 mm/min
Sensitivity Searching: 0.001 g
Velocity Measuring: 2 mm/min
Sensitivity Searching: 0,001 g
Immersion depth: 3.00 mm
Reversing distance: 10%
Values: 5-max. 20
Date acquisition: linear

The density of the oil phase is determined using a calculation method, which is outlined below:

For each individual oil contained in the formulation the density dᵢ was determined in the following manner:

The Density measurements were carried out under following conditions:
Method: Density (Triple determination)
Instrument: Densitymeter DMA 4500 (Anton Paar)
Correction: yes: for Viscosity <700 mPas
System: flexural resonator
Temperature: 20° C.

To determine the density of the total oil phase D, the following calculation is performed:

$$D = 100 / ( {\sum }_{i}^{n} ( {x}_{i} / {d}_{i} ) )$$

Wherein xᵢ is the mass fraction of each individual oil i, and dᵢ is the density of the individual oil, wherein i is an integer ranging from 1 to n and n corresponds to the total number of individual oils in the oil phase.

According to the invention the oil phase does not comprise a silicone oil.

Generally various oil compounds may be mixed to obtain the parameters as described by claim 1. This can be seen by the example formulations, which comprise oil phases which are quite different, while these oil phases are optimized to have a specific interfacial tension and density as described above.

According to the invention some oils may preferably be contained in the formulations according to the invention.

Preferred oils according to the invention are ester oils, in particular ethylhexyl stearate, ethylhexyl cocoate, isodecyl neopentanoate, caprylic/capric triglyceride, cocoglycerides, isopropyl palmitate, dibutyl adipate, isoamyl laurate, isoamyl cocoate, propylene glycol dicaprylate/dicaprate, coco-caprylate/caprate, dicaprylyl carbonate, propylheptyl caprylate, decyl oleate, c12-15 alkyl benzoate and/or decyl cocoate.

Further it is preferred if natural oils are contained. Preferred natural oils are coconut oil, (sweet) almond oil, walnut oil, peach kernel oil, apricot kernel oil, avocado oil, tea tree oil, soybean oil, glycine soja oil, sesame oil, sunflower oil, tsubaki oil, evening primrose oil, rice bran oil, palm kernel oil, mango kernel oil, cuckoo flower oil, thistle oil, macadamia nut oil, grape seed oil, amaranth seed oil, argan oil, bamboo oil, olive oil, wheat germ oil, pumpkin seed oil, mallow oil, hazelnut oil, safflower oil, canola oil, sasanqua oil, jojoba oil, rambutan oil, cocoa butter, vegetable oil and shea butter. Glycine soja oil and vegetable oil are preferred.

Further preferred oils are selected from ether oils, in particular dicaprylyl ether.

Further it is preferred if the quantity of oils that are liquid under normal condition is at least 80% by weight, more preferably 85% by weight, more preferably 90% by weight, more preferably 95% by weight, more preferably 98% by weight and most preferably at least 99% by weight, calculated to the total weight of the oil phase.

### EXAMPLES

The following examples should illustrate the compositions of this invention, without intending to limit the invention to these examples. The numerical values in the examples are percentages by weight, based on the total weight of each phase of the preparations.

The experimental results are indicated in the following table containing example formulations. All formulations designated with Com. X are reference example not according to the invention, whereby X is an integer. All formulations designated with Ex. Y are examples according to the invention, whereby Y is an integer.

| | | Ex. | Ex. | Ex. | Com. | Com. | Com. |
|---|---|---|---|---|---|---|---|
| | Ingredients | 1 | 2 | 3 | 1 | 2 | 3 |
| Water phase: | Sodium chloride | 0.2 | 0.3 | 0.3 | 0.3 | 0.2 | 0.2 |
| | Water | 95.4 | 94.9 | 92.4 | 93.0 | 95.2 | 93.7 |
| | Glycerin | 2.4 | 2.8 | 5.3 | 4.6 | 2.5 | 4.2 |
| | Benzethonium chloride | 0.1 | 0.1 | 0.1 | 0.1 | 0.1 | 0.1 |
| | Phenoxyethanol | 0.4 | 0.4 | 0.5 | 0.5 | 0.5 | 0.5 |
| | EDTA | 1.4 | 1.4 | 1.5 | 1.4 | 1.5 | 1.3 |
| | Sodium Cocoamphoacetate | 0.1 | | | | | |
| | Coco Glucoside | | | | | | 0.1 |
| | Caprylyl/Capryl Glucosie | | 0.1 | | 0.2 | 0.0 | |
| Oil phase: | Isodecyl Neopentanoate | | 2.7 | 29.0 | | | |
| | Caprylic/Capric Triglyceride | | | | | 71.6 | 53.9 |
| | Cocoglycerides | | | | 19.3 | | |
| | Isopropyl Palmitate | | | | | 9.1 | |
| | Glycine Soja Oil | | | | | 10.3 | |
| | Dibutyl Adipate | | | | 14.3 | | |
| | Vegetable Oil | | | | 25.0 | | |
| | Isoamyl Laurate | | 34.1 | | 26.3 | | |
| | Isoamyl Cocoate | | | | | | 10.9 |
| | Propylene Glycol | | 22.2 | | | | 3.4 |
| | Dicaprylate/Dicaprate | | | | | | |
| | Coco-Caprylate/Caprate | 1.5 | | | | | |
| | Dicaprylyl Ether | 5.3 | | | | | |
| | Dicaprylyl Carbonate | | | | 7.4 | | |
| | Propylheptyl | 23.5 | | | | | |
| | Caprylate + Tocopherol | | | | | | |
| | Hydrogenated Polydecene | 49.4 | 26.4 | | | | 13.2 |
| | Decyl Oleate | | 4.5 | 11.3 | 7.6 | | |
| | Ethylhexyl Stearate | 11.1 | | 13.9 | | | |
| | Ethylhexyl Cocoate | 9.2 | 10.2 | 12.8 | | 9.1 | |
| | C12-15 Alkyl Benzoate | | | 4.7 | | | |
| | Decyl Cocoate | | | 28.3 | | | 18.6 |
| Mix Quantity | Water phase | 85 | 85 | 85 | 85 | 85 | 85 |
| (v/v) | Oil phase | 15 | 15 | 15 | 15 | 15 | 15 |
| Parameter | | | | | | | |
| | Calculated Density (Oil Phase) | 0.84 | 0.86 | 0.86 | 0.90 | 0.93 | 0.81 |
| | g/cm3 | | | | | | |
| | Interfacial Tension (mN/m) | 34.0 | 29.0 | 24.9 | 20.4 | 24.8 | 22.7 |
| Observation | | | | | | | |
| (1 - yes, 0- | Haziness at Tsp (binary) | 1 | 1 | 1 | 1 | 1 | 1 |
| No) | Haziness at 24 H(binary) | 0 | 0 | 0 | 1 | 1 | 1 |
| | Separation Time (tsp) (min) | 40 | 7 | 10 | 120 | 10 | 60 |
| | Sweating: 24 H (binary) | 0 | 0 | 0 | 1 | 1 | 1 |

The examples were analyzed in the following manner. After preparation of the samples the two-phase products were shake thoroughly for 10 second. Afterwards it was waited for a full separation of the phases (time stamp called tsp). The tsp. was noted. At this time point the haziness of the phases were analyzed by optical analysis. Further, 24 h after shaking the haziness of the phases and the appearance of sweating effect (oil droplets cling to plastic bottle surface in aqueous phase) was visually inspected. If a value was determined with a yes, the value 1 was added to the table above. For the case that no haziness was observed, or no sweating was observed a 0 was entered in the table.

As indicated by the table above superior results were obtained with the formulations according to the invention.

1.-14. (canceled)

15. A two-phase cosmetic cleansing composition, wherein the composition is not an emulsion and comprises
(a) an aqueous phase, and
(b) an oil phase
having an interfacial tension towards water of from 22 mN/m to 35 mN/m
having a density of from 0.82 g/cm³ to 0.90 g/cm³, and
being free from silicone oils and lower alkanes.

16. The composition of claim 15, wherein a ratio by volume between the aqueous phase and the oil phase is from 20:1 to 1:20.

17. The composition of claim 15, wherein a ratio by volume between the aqueous phase and the oil phase is from 10:1 to 1:2.

18. The composition of claim 15, wherein a ratio by volume between the aqueous phase and the oil phase is from 8:1 to 2:1.

19. The composition of claim 15, wherein the aqueous phase comprises water in a total concentration of at least 60% by weight, based on a total weight of the aqueous phase.

20. The composition of claim 15, wherein the aqueous phase comprises water in a total concentration of less than 99% by weight, based on a total weight of the aqueous phase.

21. The composition of claim 15, wherein the aqueous phase comprises one or more surfactants.

22. The composition of claim 21, wherein a total concentration of the one or more surfactants ranges from 0.01% to 2% by weight, based on a total weight of the aqueous phase.

23. The composition of claim 21, wherein the one or more surfactants comprise one or more nonionic surfactants.

24. The composition of claim 23, wherein the one or more nonionic surfactants comprise alkyl glucosides.

25. The composition of claim 24, wherein the one or more nonionic surfactants comprise one or more of coco glucoside, caprylyl/capryl glucoside, decyl glucoside, lauryl glucoside.

26. The composition of claim 15, wherein the aqueous phase comprises one or more of phenoxyethanol, ethylhexylglycerin, and benzethonium chloride.

27. The composition of claim 15, wherein the oil phase comprises at least one ester oil.

28. The composition of claim 27, wherein the oil phase comprises one or more of ethylhexyl stearate, ethylhexyl cocoate, isodecyl neopentanoate, caprylic/capric triglyceride, cocoglycerides, isopropyl palmitate, dibutyl adipate, isoamyl laurate, isoamyl cocoate, propylene glycol dicaprylate/dicaprate, coco-caprylate/caprate, dicaprylyl carbonate, propylheptyl caprylate, decyl oleate, c12-15 alkyl benzoate, decyl cocoate.

29. The composition of claim 15, wherein the oil phase comprises at least one natural oil.

30. The composition of claim 29, wherein the oil phase comprises one or more of coconut oil, sweet almond oil, walnut oil, peach kernel oil, apricot kernel oil, avocado oil, tea tree oil, soybean oil, glycine soja oil, sesame oil, sunflower oil, tsubaki oil, evening primrose oil, rice bran oil, palm kernel oil, mango kernel oil, cuckoo flower oil, thistle oil, macadamia nut oil, grape seed oil, amaranth seed oil, argan oil, bamboo oil, olive oil, wheat germ oil, pumpkin seed oil, mallow oil, hazelnut oil, safflower oil, canola oil, sasanqua oil, jojoba oil, rambutan oil, cocoa butter, vegetable oil, shea butter.

31. The composition of claim 15, wherein the oil phase comprises at least one ether oil.

32. The composition of claim 31, wherein the oil phase comprises dicaprylyl ether.

33. The composition of claim 15, wherein a concentration of one or more oils that are liquid under normal conditions is at least 80% by weight, based on a total weight of the oil phase.

34. The composition of claim 15, wherein the aqueous phase comprises sodium chloride.
